(19) United States
(12) Patent Application Publication
(10) Pub. No.: US 2012/0276189 A1
(43) Pub. Date: Nov. 1, 2012

(54) Therapeutic Treatment of Dermatologic Skin Disorders

(76) Inventor: Benjamin Johnson, Evergreen, CO (US)

(21) Appl. No.: 11/877,614

(22) Filed: Oct. 23, 2007

(51) Int. Cl. A61K 9/127; A61P 35/00; A61K 31/716; A61P 17/02

(52) U.S. Cl. 424/450; 514/54

(57) ABSTRACT

β-glucans are incorporated with liposomes in therapeutic compositions for topical application to prevent or alleviate the conditions and symptoms of cosmetic and dermatologic disorders. In particular, β-glucan is combined with liposomes to increase the penetration of the β-glucan into the skin, to provide hydration of the skin during treatment. The deeper penetration of the β-glucan increases macrophage activity to remove scar tissue, dead skin cells and other foreign debris as well as stimulating collagen growth to improve skin health.

### FIELD OF THE INVENTION

This invention relates to the field of treatments for dermatologic and cosmetic skin disorders.

### BACKGROUND OF THE INVENTION

There are an extensive number of skin care products used for the treatment of skin disorders that may have resulted from aging, environment damage, disease or other factors. These disorders range from age spots, wrinkles, warts, acne, eczema, keratoses, psoriasis, xerosis, aging skin, biochemical disorders within the skin and many other disorders.

These skin care products use a multitude of ingredients arranged in numerous formulations. These products have had varying amounts of successes in treating dermatologic and cosmetic skin disorders.

One product that shows promise in treating skin disorders is β-glucans. β-glucans are a group of complex polysaccharides that only contain glucose as structural components. β-glucans have known applications for enhancing immune systems, wound repair and other therapeutic activities. One group of glucans that have six-sided glucose rings connected at the 1 and 3 positions are known as Beta 1,3-D glucans (β-glucans) have particular benefits in providing therapeutic activity. Other β-glucans include glucose rings connected at the 1 and 4 positions (β(1,4)-glucans) and at the 1 and 6 positions (β(1,6)-glucans.

β-glucans are known to be activate macrophages, immune system cells that trap and destroy foreign substances such as bacteria, viruses, fungi, and parasites. β-glucans also activate NK cells, T-Cells and B-cells including some cytokines. Macrophages posses specialized receptors that interact with β-glucan. This interaction with β-glucan molecules activates the macrophage cell so the macrophage cells will then identify, engulf and destroy the foreign substances. The macrophage cells may also release substances that stimulate other cells of the immune system. β-glucans have been used primarily for anti-tumor and anti-cancer treatments and for other immune responses such as infections, parasites, bacteria, fungus and other treatments.

β-glucans are also known to stimulate collagen growth in skin layers. The β-glucan molecule stimulates fibroblasts (connective tissue cells) to produce collagen.

β-glucans occur in a soluble form (β-1,3-glucan with side chain linkage at 1,4) as well as in an insoluble form (β-1,3 glucan with or without side chain linkages at 1,6). The insoluble form of B-glucans are found in baker's yeast, seaweed, certain grains including oats and barley and certain mushrooms such as Reishi, Shiitake and Maitake while the soluble form is typically extracted from yeast (Saccharomyces cerevisiae) cell walls. The soluble glucan form is typically used in nutritional dietary supplements while soluble glucans are primarily utilized in pharmaceutical applications.

β-glucans are typically administered by injections, although oral doses have been attempted with the insoluble form. There have been a few attempts at creating a topical composition using β-glucans but these have been largely unsuccessful. Topically applied compositions that include large biopolymers such as β-glucans are only able to penetrate transdermally to a shallow depth which limits their effectiveness. The β-glucan molecules pass through spacing between the skin cells in order to penetrate the dermis. Since the β-glucan molecules are relatively large, they typically do not easily pass through the skin layers. Thus the efficacy of previous topical compositions using β-glucans has been low.

Thus a problem exists in providing topical skin care products containing β-glucans have increased efficacy in the treatment of many dermatologic and cosmetic skin disorders.

### SUMMARY OF THE INVENTION

The present invention solves these and other problems by providing β-glucans with liposomes. The present invention may in various embodiments be used to increase the efficacy of the use of β-glucans for therapeutically and cosmetically treating many skin disorders.

A preferred embodiment of the present invention increases the efficacy of a topical skin care product by increasing the penetration of β-glucans into the skin. Compositions containing β-glucans are coated or mixed with liposomal materials. The effect of β-glucans on the skin occurs at the dermal level, so without adequate penetration, they have limited function in the skin. The liposomal β-glucans compound has been shown to greatly increase the penetration of the β-glucans which increases the efficacy of the product.

In a preferred embodiment of the present invention, the use of liposomal β-glucans in a topical application also may be effective in the treatment of basal cell carcinoma and actinic keratosis. The use of β-glucan with liposomes increase the penetration of the β-glucan to attack foreign bodies in the skin.

Another preferred embodiment of the present invention is useful in treating scar tissue that results from most common skin conditions, such as photodamage, acne, hyperpigmentation, rosacea and other conditions.

Another preferred embodiment of the present invention reduces the irritation to the skin. Compositions containing β-glucans are coated or mixed with liposomal materials to reduce the irritation to the skin. Also the additional hydration of the skin using the liposomal formulation reduces the irritation as well.

These and other features of the present invention will be evident from the ensuing detailed description of preferred embodiments and from the claims.

### DETAILED DESCRIPTION OF PREFERRED EMBODIMENTS

The present invention provides products and methods for increasing the efficacy of treating skin disorders. It is to be expressly understood that this exemplary embodiment is provided for descriptive purposes only and is not meant to unduly limit the scope of the present inventive concept. Other embodiments of the skin care products and methods of use of the present invention are considered within the present inventive concept as set forth in the claims herein. For explanatory purposes only, the skin care products and methods of use of the preferred embodiments are discussed primarily for the purposes of understanding the method of installation. It is to be expressly understood that other products and methods are contemplated for use with the present invention as well.

In accordance with the present invention, β-glucans are incorporated with liposomes in therapeutic compositions for topical application to prevent or alleviate the conditions and symptoms of cosmetic and dermatologic disorders are described as follows. In particular, β-glucans are combined with liposomes to increase the penetration of the β-glucan molecules into the skin, to provide hydration of the skin during treatment, and other embodiments and uses.

β-glucans

The present invention utilizes β-glucans and in particular β-1,3 glucans with or with side chain linkages at 1,6. The present invention is described below in descriptive embodiments utilizing β-1,3 glucans with or with side chain linkages at 1,6. However, it is to be expressly understood that other forms of β-1,3 glucans with or with side chain linkages at 1,6 are considered to be within the scope of the present invention as set forth in the claims.

The compositions of the present invention may also include other cosmetic and/or pharmaceutical agents along with the β-glucans to enhance their efficacy. Some examples of cosmetic and pharmaceutical agents are clotrimazole, miconazole, salicyclic acid, pramoxine, menthol, retinoic acid, hydrocortisone, hydrocortisone valerate, betamethasone valerate, betamethasone dipropionate, triamcinolone acetonide, fluocinonide, hydroquinone, clobetasol propionate, benzoyl peroxide, crotaminton, 5-fluorouracil, monobenzone, vitam A palmitate, vitamin E acetate and vitamin C.

Liposomes

Liposomes are microscopic spheres made from fatty materials, predominantly phospholipids. Because of their similarity to phospholipid domains of cell membranes and an ability to carry substances, liposomes can be used to protect active ingredients and to provide time-release properties in medical treatment.

Liposomes are made of molecules with hydrophilic and hydrophobic ends that form hollow spheres. They can encapsulate water-soluble ingredients in their inner water space, and oil-soluble ingredients in their phospholipid membranes. Liposomes are made up of one or more concentric lipid bilayers, and range in size from 50 nanometers to several micrometers in diameter. Liposomal formulations have been used for many years to enhance the penetration of topically applied ingredients. Liposomes are made from lecithin, egg or it can be synthesized. These phospholipids can be both hydrogenated and non-hydrogenated. Phosphatidylcholine is extracted from these sources and can be both saturated and unsaturated. Other phospholipids including essential fats like linoleic acid and alpha linolenic acid can be used. Additionally, polyethylene glycol and cholesterol are considered liposomal material because of their lipid structure.

Preparation of Exemplary Therapeutic Compositions

Accordingly, a preferred embodiment of the present invention provides cosmetic as well as medicinal compositions containing β-glucans coated in liposomal material which when topically administered will substantially improve and alleviate the symptoms of various cosmetic conditions or dermatologic disorders.

β-glucans of the instant invention may be formulated for topical application in aqueous or non-aqueous solution, gel, lotion, cream or ointment containing 0.1 to 20 percent and preferably from 0.5 to 15 percent by weight of the total composition. Other additives may be used to stabilize or otherwise provide functionality such as solvents, plasticizers, emulsify, stiffen, or other functions. Liposomal lecithin or a liposome substitute or other lipid preparations are added to the above solution with mixing until a uniform consistency is obtained.

To prepare a typical aqueous solution, β-glucans are dissolved in a mixture of water, ethanol and propylene glycol in a volume ratio of 30:50:20, respectively. Sodium metabisulfite is then added to the above solution. Liposomes such as lecithin or phosphatidylcholine or other lipid preparations are added to the above solution with mixing until a uniform consistency is obtained.

To prepare a typical non-aqueous solution, β-glucans are dissolved in a mixture of ethanol, isopropyl myristate and squalane in a volume ratio of 70:20:10, respectively. BHT is then added to the above solution. Liposomes or liposome substitutes are added to this solution with mixing until a uniform consistency is achieved. When a combination composition is desired retinyl palmitate and/or hydroquinone, for example is added to the above non-aqueous solution. The preferred concentration of retinyl palmitate ranges from 1 to 5%. The concentration of hydroquinone may range from 1 to 5%, but the preferred concentration is 2% by weight of the total composition.

A typical cream or lotion containing β-glucans is prepared by first dissolving β-glucans in ethanol, acetone, propylene glycol or other solvent. The solution thus prepared is then admixed with commonly available oil-in-water emulsions. BHT or sodium metabisulfite may be added to such emulsions to stabilize the β-glucans. Liposomes or liposome substitutes are added to this solution with mixing until a uniform consistency is achieved.

A typical gel composition is formulated by first dissolving β-glucans in a mixture of ethanol, water and propylene glycol in a volume ratio of 50:30:20, respectively. A gelling agent such as hydroxyethylcellulose, hydroxypropylcellulose or hydroxypropylmethylcellulose is then added to the mixture with mixing. The preferred concentration of the gelling agent may range from 0.2 to 2 percent by weight of the total composition. Liposomes or liposome substitutes are added to this solution with mixing until a uniform consistency is achieved.

The above examples of formulations and compositions of descriptive embodiments are provided as a general explanation of the present invention. It is expressly noted that these examples are intended to be illustrative and not limiting.

Therapeutic Uses

The present invention may in various embodiments be used to increase the efficacy of the use of β-glucans 1 for therapeutically and cosmetically treating many skin disorders.

A preferred embodiment of the present invention increases the efficacy of a topical skin care product by increasing the penetration of β-glucans into the skin. Compositions containing β-glucans are coated or mixed with liposomal materials as described above. The liposomal β-glucans compound has been shown to increase the penetration of the β-glucans increases the efficacy of the β-glucans product.

The β-glucans then activate macrophages in the skin layers to help remove scar tissue, dead skin cells and other debris in the skin layers. The β-glucans also stimulate collagen growth. This is critical in improving the look and health of skin that is damaged as part of the aging process and from external exposure to ultraviolet rays, smoking, free radicals and other factors. The preferred embodiment of the liposomal β-glucans allow deeper penetration of this beneficial activity that has not been possible in prior topical compositions.

The effect of β-glucans on the skin occurs at the dermal level, so without adequate penetration, they have limited function in the skin. The liposomal β-glucans compound has been shown to greatly increase the penetration of the β-glucans which increases the efficacy of the product.

In a preferred embodiment of the present invention, the use of liposomal β-glucans in a topical application also may be effective in the treatment of basal cell carcinoma and actinic keratosis. The use of β-glucan with liposomes increase the penetration of the β-glucan to attack foreign bodies in the skin.

Another preferred embodiment of the present invention is useful in treating scar tissue that results from most common skin conditions, such as photodamage, acne, hyperpigmentation, rosacea and other conditions. The topical application of β-glucan will remove the scar tissue from the damaged skin.

Another preferred embodiment of the present invention increased the hydration of the skin from using β-glucans. Compositions containing β-glucans are coated or mixed with liposomal materials as described above. Liposomes have a natural affinity for water which assists in increasing the moisture in the skin during topical application of the liposomal β-glucans composition.

The invention may be embodied in other specific forms without departing from the spirit or essential characteristics thereof. The present embodiments are therefore to be considered in all respects as illustrative and not restrictive, the scope of the invention being indicated by the appended claims and all changes which come within the meaning and equivalency of the claims are therefore intended to be embraced therein.

What is claimed is:

1. A method for increasing the transdermal penetration of β-glucans compounds from a topical skin care application, said method comprising the steps of:
providing a β-glucan compound in the skin care application; and
providing said β-glucan compound within a liposome compound to increase the transdermal penetration of said β-glucan compound.

2. The method for increasing the transdermal penetration providing β-glucan from a topical skin care application of claim 1 wherein step of providing said β-glucan compound includes:
providing β-glucan having linkages at 1 and 3.

3. The method for increasing the transdermal penetration of β-glucan from a topical skin care application of claim 1 wherein step of providing said β-glucan compound includes:
providing β-glucan having linkages at 1 and 3 and side linkages at 1 and 6.

4. The method for increasing the transdermal penetration of β-glucan from a topical skin care application of claim 1 wherein step of providing said β-glucan compound includes:
providing said β-glucan compound in the range of between 0.1 percent to 20 percent by weight.

5. The method for increasing the transdermal penetration of β-glucan from a topical skin care application of claim 1 wherein step of providing said β-glucan compound includes:
providing lipid compounds having enhanced penetration properties.

6. The method for increasing the transdermal penetration of β-glucan from a topical skin care application of claim 1 wherein step of providing said β-glucan compound includes:
providing said liposome compound in the range of 0.1 percent to 20 percent by weight.

7. The method for increasing the transdermal penetration of β-glucan from a topical skin care application of claim 1 wherein step of providing said β-glucan compound includes:
providing a lipid compound having a high affinity for water.

8. A topical composition for therapeutic treatment of skin, said composition comprising:
a liposomal compound; and
a β-glucan compound contained within said liposomal compound.

9. The topical composition of claim 8 wherein said β-glucan compound includes:
β-glucan having linkages at 1 and 3.

10. The topical composition of claim 8 wherein said β-glucan compound includes:
β-glucan having linkages at 1 and 3 and side linkages at 1 and 6.

11. The topical composition of claim 8 wherein said composition includes:
β-glucan compound in a range of between 0.1 and 20 percent by weight.

12. The topical composition of claim 8 wherein said composition includes:
said liposomal compound has a high affinity for water.

13. A method for removing scar tissue from damaged skin, said method comprising:
providing a liposomal compound;
providing a β-glucan compound contained within said liposomal compound;
applying said β-glucan compound contained within said liposomal compound to the damaged skin.

14. The method for removing scar tissue from damaged skin of claim 13 wherein step of providing said β-glucan compound includes:
providing β-glucan having linkages at 1 and 3.

15. The method for removing scar tissue from damaged skin of claim 13 wherein step of providing said β-glucan compound includes:
providing β-glucan having linkages at 1 and 3 and side linkages at 1 and 6.

16. The method for removing scar tissue from damaged skin of claim 13 wherein step of providing said β-glucan compound includes:
providing said β-glucan compound in the range of between 0.1 percent to 20 percent by weight.

17. A method for treating basal cell carcinoma, said method comprising:
providing a liposomal compound;
providing a β-glucan compound contained within said liposomal compound;
applying said β-glucan compound contained within said liposomal compound to the basal cell carcinoma in the skin.

18. The method for treating basal cell carcinoma of claim 17 wherein step of providing said β-glucan compound includes:
providing β-glucan having linkages at 1 and 3.

19. The method for treating basal cell carcinoma of claim 17 wherein step of providing said β-glucan compound includes:
providing β-glucan having linkages at 1 and 3 and side linkages at 1 and 6.

20. The method for treating basal cell carcinoma of claim 17 wherein step of providing said β-glucan compound includes:
providing said β-glucan compound in the range of between 0.1 percent to 20 percent by weight.
